(19) United States
(12) Patent Application Publication
(10) Pub. No.: US 2025/0276010 A1
(43) Pub. Date: Sep. 4, 2025

(54) MATERIALS CONTAINING COPPER AND THEIR USE IN BENEFICIAL BODY HEALTH

(71) Applicant: FINE COTTON FACTORY INC., Toronto (CA)

(72) Inventors: Biren PATEL, Toronto (CA); Gary SWEENEY, Toronto (CA); Erfei SONG, Guangzhou (CN); Amy BOTTA, Toronto (CA)

(21) Appl. No.: 19/210,557

(22) Filed: May 16, 2025

Related U.S. Application Data
(63) Continuation-in-part of application No. 17/271,869, filed on Feb. 26, 2021, now abandoned.
(60) Provisional application No. 62/723,235, filed on Aug. 27, 2018.

(51) Int. Cl. A61K 33/34; A61K 9/70; A61P 3/10

(52) U.S. Cl. CPC A61K 33/34; A61K 9/7007; A61P 3/10

(57) ABSTRACT

Methods of managing body weight, increasing insulin sensitivity, promoting cardiovascular health and increasing the amount of beneficial bacteria include the step of exposing a subject to an article containing copper and their use in beneficial body health.

### 1. FIELD OF THE INVENTION

The present invention relates to materials containing copper and their use in beneficial body health.

### 2. BACKGROUND OF INVENTION

### Body Weight

Currently just over a third of men are overweight or obese, and nearly 40% of women are overweight or obese (Ng M, Fleming T, Robinson M et al. Global, regional and national prevalence of overweight and obesity in children and adults during 1980-2013: A systematic analysis for the Global Burden of Disease Study 2013. Lancet 2014; 384 (9945); 766-81).

The causes of obesity are as varied as the people it affects. At its most basic, of course, obesity results when an energy imbalance occurs due to a difference in calories consumed and calories expended. The body stores these excess calories as body fat, and over time the extra pounds add up. Eat fewer calories than the body burns, weight goes down. This equation can be deceptively simple, though, because it does not account for the multitude of factors that affect what we eat, how much we exercise, and how our bodies process all this energy.

The health consequences associated with overweight and obesity are numerous and at the rate they are progressing the economic burdens will begin to cripple many countries in the near future. For example, 44% is attributed to the diabetes burden, 23% of the heart disease burden and between 7% and 41% of certain cancer burdens are attributable to overweight and obesity.

The following are the major risks: breathing disorders (e.g., sleep apnea, chronic obstructive pulmonary disease); certain types of cancers (e.g., prostate and bowel cancer in men, breast and uterine cancer in women); coronary artery (heart) disease; depression; diabetes; gallbladder or liver disease; gastro esophageal reflux disease (GERD); high blood pressure; high cholesterol; joint disease (e.g., osteoarthritis); stroke.

Aside from the medical complications, obesity is also linked to psychosocial problems such as low self-esteem, discrimination, difficulty finding employment, and reduced quality of life, thus leading to depression, eating disorders and crash diets.

There are hundreds of diets, diet aids, supplements and vitamins, exercise and lifestyle experts located all over the world that are trying to help eradicate this global epidemic. However what seems to be such a simple concept of eat less and exercise more will equate to weigh less and be healthy does not seem to be achievable for the general masses of the world.

Weight obesity and its causes have, in many ways, become woven into the fabric of our society. To successfully disentangle them will take a multifaceted approach that not only gives individuals the skills to make healthier choices but also sets in place policy and infrastructure that support those choices.

Although the health risks are modifiable or preventable there are very little efforts in progress in the majority of the developing world to control them. There needs for a simple article and method of controlling body weight gain that does not include the use drugs, diets or exercise or invasive treatments.

### Insulin Sensitivity

With the establishment of therapy with insulin in insulin-deficient diabetes in humans the complications of long-term diabetes mellitus were recognized in what is now termed Type 1 diabetes (T1D). It was later recognized that similar complications occur in long-term maturity onset diabetes, now known as Type 2 diabetes (T2D). The metabolic control achievable with exogenous insulin in these early decades following discovery of the hormone did not restore blood glucose levels to the normal range and it was speculated that long-term hyperglycaemia had a mechanistic role in the development of the microvascular and macrovascular complications associated with long-term diabetes.

T2D is well recognized as generally developing at a later age. It is associated with obesity in many subjects. However, it was inferred that benefits of intensive insulin therapy (IIT) would be demonstrable in people with phenotypic T2D (N Engl J Med 1993; 329(14):977-986). It is clear that a very high proportion of people with long-term phenotypic T2D (more than 75%) eventually develop hyperglycaemia of a degree that calls for insulin treatment, and this has become the standard progression in treatment of T2D with diet, oral hypoglycaemic agents, and later as glycaemic control fails, with addition of administration of insulin according to the same programs used in T1D. Thus IIT is indicated in clinical practice for the management of T2D when clinical-guideline-approved targets for glycaemic control are not attainable without administration of insulin. The adverse effects of IIT remain prominent obstacles to its optimal use in T1D or T2D. It is clear that the major adverse effects of IIT, i.e. intermittent hypoglycaemia, and progressive weight gain, constitute an obstacle to the acceptance of IIT. These risks and the wide-spread fear of them are responsible for reluctance on the part of physicians as well as patients to initiate IIT in T2D, where it is optional, rather than obligatory as it is in T1D. There needs for a simple article and method of improving insulin sensitivity while controlling body weight gain that does not include the use drugs, diets or exercise or invasive treatments.

### Protection of Heart Function

Cardiovascular diseases account for 12 million deaths annually worldwide and myocardial infraction (MI) is a leading cause of morbidity and mortality. Cardiac rupture, ventricular arrhythmia and heart failure are common causes of morbidity and mortality following MI. Cardiac rupture refers to a rupture of the left ventricle of the heart, generally following an acute myocardial infarction. Left untreated, the condition usually is fatal immediately or within a few days depending on the extent of the rupture. It is believed that such rupture occurs in approximately 10% of patients with fatal acute myocardial infarction [Bates, RJ, Am J Cardiol. 40:429-3]. Myocardial rupture causes 25,000 deaths a year in the United States alone and is the second most common cause of the death after an acute myocardial infarction. There is a need, therefore, for a simple article and method of improving heart function as a means to protect the heart and prevent cardiovascular disease that does not include the use of drugs, diets, exercise or invasive treatments.

### Gut Microbiota

Mammals harbor a complex gut-associated microbiota, comprising bacteria that provide immunological, metabolic and neurological benefits to the host, and contribute to their wellbeing. However, dysregulation of the microbiota composition, known as dysbiosis, plays a key role in the pathogenesis of many diseases, including inflammatory bowel diseases, type 1 and type 2 diabetes, asthma, multiple sclerosis, Hashimoto's disease, other autoimmune disorders and so forth. There is a need, therefore, for a simple article and method of improving and protecting gut flora that does not include the use of drugs, diets, exercise or invasive treatments.

Interestingly, copper has been shown in the past to have anti-microbial properties. However, it has not been shown that external exposure to articles containing copper would affect the guts flora.

### 3. SUMMARY OF THE INVENTION

The present invention relates to articles or agents containing copper that provide benefits to body health. The present invention provides in part quality of life (QOL) improving or sustaining articles containing copper, for example, a physical health improving or sustaining copper-containing article or agent.

In one embodiment, the present invention provides a quality of life (QOL) improving or sustaining method for a human by contacting the human with an article containing copper.

In one embodiment, the present invention provides for a method for promoting the growth of beneficial bacteria in the gastro-intestinal tract of a subject by contacting the subject with an article containing copper, where the article containing copper includes between 0.05% to 4.5% copper by weight and is capable of releasing copper ions, and where the release of copper ions promotes the growth of beneficial bacteria in the gastro-intestinal tract of the subject.

In one embodiment of the method for promoting the growth of beneficial bacteria in the gastro-intestinal tract of a subject of the present invention, the beneficial bacteria are probiotic bacteria.

In another embodiment of the method for promoting the growth of beneficial bacteria in the gastro-intestinal tract of a subject of the present invention, the beneficial bacteria include Lactobacillus sp. (LB), Clostridium coccoides sp. (CC), and Segmented filamentous bacteria (SFB).

In another embodiment of the method for promoting the growth of beneficial bacteria in the gastro-intestinal tract of a subject, the article containing copper is a liquid treated article, where the liquid contains copper.

In another embodiment of the method for promoting the growth of beneficial bacteria in the gastro-intestinal tract of a subject of the present invention, the article containing copper, is a copper infused article.

In another embodiment, the present invention provides a method of managing the body weight of a subject by contacting the subject with an article containing copper where the article containing copper includes between 0.05% to 4.5% copper by weight and is capable of releasing copper ions and where the release of copper ions manages the body weight of the subject.

In one embodiment of the method of managing body weight of a subject of the present invention, the method further includes the steps of weighing and recording a body weight of the subject before and after exposure to the article containing copper, and comparing the body weight before and after exposure.

In another embodiment, the present invention provides a method for improving or increasing insulin sensitivity in a subject in need of such therapy, by contacting the subject with an article containing copper where the article containing copper includes between 0.05% to 4.5% copper by weight and is capable of releasing copper ions and where the release of copper ions results in the desired effect with respect to insulin sensitivity. In one aspect of this embodiment the subject suffers from type 2 diabetes. In another aspect of this embodiment, the subject suffers from type 1 diabetes.

In another embodiment, the present invention provides a method for promoting, treating and/or prophylaxis of cardiovascular health in a subject, by contacting the subject with an article containing copper where the article containing copper includes between 0.05% to 4.5% copper by weight and is capable of releasing copper ions and where the release of copper ions results in the desired effect with respect to cardiovascular health. In one aspect of this embodiment the subject is a subject in need of such therapy.

In another embodiment, the present invention provides a method of increasing the amount of adiponectin in blood, the method comprising the step of exposing a subject to an article containing copper.

In another embodiment, the present invention provides a method of increasing the amount of FGF-21 in blood, the method comprising the step of exposing a subject to an article containing copper.

In another embodiment, the present invention provides a method of reducing the amount of lipocalin-2 in blood, the method comprising the step of exposing the subject to an article containing copper.

In another embodiment, the present invention provides a method for increasing the amount of adiponectin or FGF-21, or of reducing the amount of lipocalin-2, in blood, by contacting a subject with an article containing copper where the article containing copper includes between 0.05% to 4.5% copper by weight and is capable of releasing copper ions, and where the release of copper ions increases the amount of adiponectin or FGF-21, or reduces the amount of lipocalin-2, in the blood of the subject.

In one embodiment according to any one of the previous embodiments, the subject is a human.

In one embodiment according to any one of the previous embodiments, the article containing copper, is a liquid treated article, wherein the liquid contains copper.

In one embodiment according to any one of the previous embodiments, the article containing copper, is a copper infused article.

In one embodiment, the present invention provides a use of an article in promoting the growth of beneficial bacteria in the gastro-intestinal tract of a subject, wherein the article contains copper.

In one embodiment of the present invention the beneficial bacteria are probiotic bacteria.

In another embodiment of the present invention the beneficial bacteria include Lactobacillus sp. (LB), Clostridium coccoides sp. (CC), and Segmented filamentous bacteria (SFB).

In another embodiment of the present invention, the article containing copper, is a liquid treated article, wherein the liquid contains copper.

In another embodiment of the present invention, the article containing copper, is a copper infused article.

In one embodiment, the present invention provides for a use of an article in managing body weight of a subject, wherein the article contains copper.

In one embodiment, the present invention provides for a use of a copper-containing article in promoting, treating and/or prophylaxis of cardiovascular health in a subject.

In one embodiment, the present invention provides for a use of a copper-containing article in improving or increasing insulin sensitivity in a subject in need.

In one embodiment, the present invention provides for a use of a copper-containing for increasing the amount of adiponectin in blood of a subject.

In one embodiment, the present invention provides for a use of a copper-containing article for increasing the amount of FGF-21 in blood of a subject.

In one embodiment, the present invention provides for a use of a copper-containing article for reducing the amount of lipocalin-2 in blood of a subject.

In another embodiment, the present invention provides a use of an article containing infused copper, where the article containing infused copper comprises between 0.05% to 4.5% copper by weight and is capable of releasing copper ions, and where the release of copper ions increases the amount of adiponectin or FGF-21, or reduces the amount of lipocalin-2, in the blood of a subject.

In one embodiment of the present invention the subject is a human.

In another embodiment of the present invention, the copper-containing article, is a liquid treated article, wherein the liquid contains copper.

In another embodiment of the present invention, the copper-containing article, is a copper infused article.

In another embodiment, the copper-containing article according to any of the previous embodiments, is an article of manufacture.

In another embodiment, the copper-containing article according to any of the previous embodiments, is a fabric. In one embodiment, the fabric may include a single layer. In other embodiments, the fabric may include 2 or more layers, where the copper is contained in a layer that is not in contact with the subject. In other embodiments, the fabric may include 3 or more layers, the three or more layers including a face layer, an inlay and a back layer, where the inlay and the back layer are not in contact with the subject, and where the copper is contained in the inlay or the back layer that is not in contact with the subject. In other embodiments, the fabric may be a sheet, top of the bed product, mattress and mattress covers, removable mattress toppers, ticking fabric for covering a mattress pillow cases, pillow shell, pillow fill, blanket and blanket fill, fitted and loose sheets for bedding, mattress pads, chair liners, clothing, towels or sleeping bag.

In another embodiment, the copper-containing article according to any of the previous embodiments contains an effective amount of copper.

### 4. BRIEF DESCRIPTION OF THE FIGURES

The present invention will become more fully understood from the detailed description given herein and from the accompanying drawings, which are given by way of illustration only and do not limit the intended scope of the invention.

FIG. 1 is a graph illustrating changes in body weight with time in control mice (black/dark bars) and mice exposed to/treated with the copper-containing articles of the present invention (grey bars).

FIGS. 2A to 2D are graphs illustrating glucose tolerance tests (GTT) at 4 weeks (FIG. 2A) and 8 weeks (FIG. 2C) and insulin tolerance tests (ITT) at 4 weeks (FIG. 2B) and 8 weeks (FIG. 2D) in control mice and mice exposed to/treated with the copper-containing articles of the present invention. The ITT is technically very similar to the GTT as it involves monitoring blood glucose levels over time, but in response to insulin administration. Control=black/dark bars; exposed to/treated with copper-containing articles: grey bars.

FIGS. 3A and 3B are echocardiography graphs on mice exposed to/treated with a copper-containing article of the present invention and controls at 4 weeks (FIG. 3A) and 8 weeks (FIG. 3B). Control mice=black/dark bars; mice exposed to/treated with copper-containing articles: grey bars.

FIG. 4 is a graph illustrating relative abundance of bacteria in stool of control mice and mice exposed to/treated with a copper-containing article of the present invention. (BacF—Bacteroides fragilis; BacS—Bacteroides sp. LB—Lactobacillus sp; CC—Clostridium coccoides sp.; SFB—Segmented filamentous bacteria —; EntB—Enterobacteriaceae; ER—Eubacterium rectale). Control mice=black/dark bars; mice exposed to/treated with copper-containing articles: grey bars.

FIG. 5A is a graph illustrating the amount (ng/ml) of lipocalin-2 in blood in control mice and mice exposed to/treated with a copper-containing article of the present invention. Control mice=black/dark bars; mice exposed to/treated with copper-containing articles: grey bars.

FIG. 5B is a graph illustrating the amount (pg/ml) of FGF-21 in blood in control mice and mice exposed to/treated with a copper-containing article of the present invention. Control mice=black/dark bars; mice exposed to/treated with copper-containing articles: grey bars.

FIG. 5C is a graph illustrating the amount (ng/ml) of adiponectin in blood in control mice and mice exposed to/treated with a copper-containing article of the present invention. Control mice=black/dark bars; mice exposed to/treated with copper-containing articles: grey bars.

FIG. 6: illustration of a knitting sequence of a copper-containing fabric according to one embodiment of the present invention.

FIG. 7: illustration of a knitting sequence of a copper-containing fabric according to another embodiment of the present invention.

FIG. 8: illustration of a knitting sequence of a copper-containing fabric according to another embodiment of the present invention.

FIG. 9: illustration of a knitting sequence of a copper-containing fabric according to another embodiment of the present invention.

### 5. DETAILED DESCRIPTION OF THE INVENTION

### 5.1 Overview

The present invention discloses the use of copper-containing articles used for controlling body weight, improving insulin sensitivity, enhancing the presence of beneficial flora in the gastro-intestinal tract, and improving heart function. In one embodiment of the present invention the copper may be provided in textiles or fabrics with health-improving properties. The textiles of the present invention may be used to manufacture a variety of articles of manufacture, including mattress ticking fabric, bedding (i.e. covering of a bed such as sheets, blankets, duvet), clothing, gloves, socks, welding bibs and blankets, blankets, linings, drapes, upholstery, seat covers, automobile interiors, pillow covers, eye covers, head-bands and the like.

The applicant has shown that copper contained in articles such as textiles, foams and fabrics can help improve metabolic and cardiovascular health conditions in subjects. The textiles or fabrics may be manufactured with copper-containing yarns, or may be infused, embedded or impregnated or liquid treated with copper. Foams may be infused, embedded, liquid treated or impregnated with copper. The copper may be in metallic form (e.g., particulates, alloys and oxides), salts and/or ionic form.

### 5.2 Definitions

Unless defined otherwise, all technical and scientific terms used herein have the same meaning as commonly understood by one of ordinary skill in the art to which this invention belongs. Also, unless indicated otherwise, except within the claims, the use of “or” includes “and” and vice versa. Non-limiting terms are not to be construed as limiting unless expressly stated or the context clearly indicates otherwise (for example “including”, “having” and “comprising” typically indicate “including without limitation”). Singular forms included in the claims such as “a”, “an” and “the” include the plural reference unless expressly stated otherwise. All relevant references, including patents, patent applications; government publications, government regulations, and academic literature, and including the priority document, are hereinafter detailed and incorporated by reference in their entireties.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

“Beneficial bacteria,” also known as “good bacteria” are defined as any bacteria that are beneficial to the body and enhance health. One of the most well-known types of good bacteria are probiotics.

“Effective amount” as used herein is meant an amount of copper content in an article, high enough to significantly positively modify the condition to be treated but low enough to avoid serious side effects (at a reasonable benefit/risk ratio), within the scope of sound medical judgment. An effective amount of the copper content is determined based on the intended goal. The quantity of copper depends on the protection or treatment desired. Thus, in some case dosages can be determined by measuring for example changes in body weight, serum insulin or glucose levels of a subject, GI tract flora, and so forth.

“Fiber” and “fibers” refer to any slender, elongated structure that can be carded or otherwise formed into a thread. Examples of fibers include “stable fibers,” a term that is well-known in the textile art.

The term “knit” may be used as a verb (to make (a fabric or garment) by interweaving yarn or thread in a series of connected loops either by hand, with knitting needles, or on a machine) or as a noun (a fabric or garment made by knitting).

As used herein, the term “substantially” includes exactly the term it modifies and slight variations therefrom.

“Yarn” refers to a structure comprising a plurality of threads.

Cardio-vascular disease or condition may include one or more of high blood pressure, (hypertension), coronary heart disease or widely known as heart attack, cardiovascular diseases (CVD), cerebrovascular disease (stroke), angina (chest pain), heart failure, peripheral arterial disease, atherosclerosis, cardiac arrhythmias and rheumatic heart disease amongst others.

### 5.3 Copper Containing Articles

The applicant has shown that copper contained in articles such as textiles, foams and fabrics articles can be used for controlling body weight, improving insulin sensitivity, enhancing the presence of beneficial flora in the gastro-intestinal tract, and improving heart function. In some embodiments, copper contained in articles such as textiles, foams and fabrics articles can increase the amount of adiponectin or FGF-21, or reduce the amount of lipocalin-2, in the blood of a subject. These effects can be explained by the release of copper ions from the inner layer(s) of an article containing copper, such as a fabric in accordance with the present disclosure (e.g., a fabric containing infused copper of between 0.05% to 4.5% copper by weight that may be infused with a solution containing 1.0-2.0 micron particles of copper in an inner layer) and the copper ions travelling through the outer layer(s) to exert the observed effects.

The textiles or fabrics may be manufactured with copper-containing yarns, or may be infused, embedded or impregnated with copper. Foams may be infused, embedded or impregnated with copper. The copper-may be in metallic form (e.g., particulates, alloys and oxides), salts and/or ionic form. In some embodiments, an article containing infused copper, such as a fabric in accordance with the present disclosure (e.g., an article containing infused copper of between 0.05% to 4.5% copper by weight) may be infused with a solution containing 1.0-2.0 micron particles of copper.

In some embodiments, an article containing infused copper, such as a fabric in accordance with the present disclosure (e.g., an article containing infused copper of between 0.05% to 4.5% copper by weight that may be infused with a solution containing 1.0-2.0 micron particles of copper) is capable of releasing copper ions. In some embodiments, a fabric in accordance with the present disclosure (e.g., a fabric containing infused copper of between 0.05% to 4.5% copper by weight) is capable of releasing copper ions. In some embodiments, the release of copper ions can be useful in controlling body weight, improving insulin sensitivity, enhancing the presence of beneficial flora in the gastro-intestinal tract, and improving heart function. In some embodiments, the release of copper ions can increase the amount of adiponectin or FGF-21, or reduce the amount of lipocalin-2, in the blood of a subject.

In one embodiment, the copper may be provided on its own. For example, a subject may be in the proximity of or in contact with copper or copper containing objects or articles, such as copper coins, cables with copper wires, naked copper wires, and so forth. By “in the proximity” is meant that the subject is not in direct contact with the copper or copper-containing article, but effectively or sufficiently close to the copper or copper-containing article to positively exert its health benefits. The proximity may vary with the duration of the exposure, the nature of any barriers between the copper-containing article and the subject, the number of barriers between the copper-containing article and the subject, the distance between the copper and the subject or any combination thereof. For example, in a multi-layer fabric, the copper may be restricted to a layer that is not in direct contact with the subject such that there would be at least one layer of fabric between the copper-containing layer and the subject. For example, the copper may be restricted to the inlay of a three-layer fabric, as such there would be at least one layer of fabric between the copper and the subject i.e., the copper is contained in the inlay layer that is not in contact with the subject. Subject to the nature of the barrier, the effective proximity to exert its heath properties will thus be the maximum distance between the copper in the article and the subject, or the maximum number of barriers between the copper and the subject which will provide the desired effect on the subject. Accordingly, a fabric in accordance with the present disclosure (e.g., an article containing infused copper of between 0.05% to 4.5% copper by weight that may be infused with a solution containing 1.0-2.0 micron particles of copper) can exert the desired effect on the subject by releasing copper ions across, for example, layers of fabric and the skin of the subject even when the copper containing layer is not in contact with the subject. In another embodiment, the subject may apply to a body surface the copper-containing article of the present invention for an effective amount of time.

The copper containing articles of the present invention were found to have a therapeutic or ameliorating effects even after a relatively short period of time of the subject being in the proximity or in contact with the copper-containing articles of the present invention. As such, the subject may sleep in the proximity of or in contact with or apply the copper containing article of the present invention for less than one day, one day, more than one day, one week, more than one week, one month, more than one month, a year or even more than one year.

The copper-containing textile or fabric of the present invention may be made using a yarn comprising a polymer or cellulose, or re-generated cellulose, or otherwise, and copper. The copper may be provided as a powder, liquid or alloy forms. Polymers include a compound suitable for fiber and fabric generation including a thermoplastic polymer, polyester, nylon, rayon, polyethylene (PE), polypropylene (PP), polyethylene terephthalate (PET), polyethylene terephthalate glycol (PETG), co-PET, polylactic acid (PLA), and polytrimethylene terephthalate (PTT).

In some embodiment, the yarn of the present invention may be copper-containing fiber. An example of a copper-containing fiber is described in WO/2012/088507, the content of which is incorporated herein by reference, which describes fibers that have been infused with copper and a dispersion liquid. Briefly, a polymer is mixed with an anti-microbial alloy powder and a dispersion liquid, resulting in a mixture. A fiber containing the anti-microbial powder is formed from the mixture. The fiber may then be processed for an intended use. According to WO/2012/088507, the anti-microbial agent in the alloy powder may be copper, which can be in metallic forms (e.g., particulates, alloys and oxides), salts (e.g., sulfates, nitrates, acetates, citrates, and chlorides), in ionic forms, and/or as an alloy powder with a particle size of less than 1 micron, and preferably 0.3 to 0.6 micron. The alloy powder may include copper and alkali metals such as lithium, sodium, potassium, magnesium, and/or calcium. The combination may comprise a binary combination, ternary combination, quaternary combination, or even higher order combination. The selected alloys, and the relative percentages of each alloy, may be selected depending on the intended use of the fiber or other selection criteria. Different combinations will result in different anti-microbial classes that may be used with the present invention. The dispersion liquid is a liquid additive used to disperse the copper and assist with the combination of the copper and the polymer. This allows for more uniform dispersion of the anti-microbial agent throughout the eventual fiber. The dispersion liquid itself is embedded in the fiber during manufacture but at least a portion of the dispersion liquid may dissolve from the fiber during treatments, or launderings.

Examples of dispersion liquids include anti-stats, anionic anti-stat oils, phosphate esters, vegetable oils, and other liquids. In one embodiment, the dispersion liquid may be comprised of predominately a phosphate ester with 10-30% water. The dispersion liquid may also be comprised of certain waxes, such as Montan Wax that operates to carry powders into fiber. The selection of the dispersion liquid may also relate to other desired characteristics of the fiber, including the desired tenacity, color, feel, etc.

The method of WO/2012/088507 describes the manufacture of fibers infused with copper, and can be referred to as “copper infused articles.”

In another embodiment, an article, such as a textile or fabric, may be run through liquid solution containing copper. The liquid solution containing copper is then squeezed out of the article and the article is then dried. The liquid solution containing copper can be a copper dispersed in a liquid. The liquid can be water or any other suitable solution. The copper content in the liquid solution may range anywhere from 0.05% Cu to 99% Cu. Examples of articles include, articles containing natural or artificial polymers, such as 100% cotton articles, cotton face/polyester fill articles, or 100% polyester articles or articles made with the polymers previously described. For convenience, this treatment will be referred to as “liquid treated articles.”

In another embodiment, the textiles or fabrics of the present invention may contain copper attached, coupled or connected to the fabric, or the copper may be loose. For example, the fabric may be in the form of a bag having copper inside the bag in loose form or attached to the inside walls of the bag, or copper may be attached, connected, or coupled to an existing fabric. Alternatively, the fabric may contain one or more pockets into which the copper can be contained in loose or attached form. The pockets may be designed to be in a closed or opened from. For example, the pocket may include attachment means such as buttons or Velcro® or may include a flap to open or close the pocket. This design of pockets may allow a person to insert or remove the copper from the pockets. Copper may be provided in any form as previously described. For example, the copper may be provided as a powder, liquid, alloys, filaments, wires, or in the form of coins (for example one cent coins known as pennies). The copper may be provided in metallic form, salt from or ionic form.

The fabric of the present invention may have one, two, three, four or more than four layers. A three layer fabric (with inlay), ticking fabric and/or double knit fabric (two layer without inlay), and/or a single knit construction of any kind, or a woven of any kind or content. The copper may be included any one of the layers of the fabric, and in more than one layer. For example, in a two layer fabric, the copper may be included in both layers, or in just one layer. In a three layer fabric having a top, middle and bottom layers, the copper may be included in just one of the 3 layers (in any of the top, middle or bottom layers), in 2 of the 3 layers (in any combination of two of the top, middle or bottom layers) or in all of the 3 layers. In some embodiments, the copper can be restricted to the internal layers of a multi-layer fabric such that the copper-containing layer is not in direct contact with a subject.

The applicant surprisingly discovered that the health benefits of copper can be achieved even when the copper is contained in a layer of a fabric that is not in direct contact with a subject, for example the inlay of a fabric in accordance with the present disclosure, such as a fabric containing infused copper of between 0.05% to 4.5% copper by weight, i.e. where the copper-containing layer of the fabric containing infused copper was not in direct contact with the subject using the fabric. Such a copper-containing layer of a fabric containing infused copper of between 0.05% to 4.5% copper by weight, is capable of releasing copper ions. By contrast, tests on other known fabrics having the copper only in the inlay (e.g., other known copper-infused fabrics) have been shown to not release copper ions. Furthermore, a fabric in accordance with the present disclosure, such as a fabric containing 30% of copper-infused yarn, for example containing between 0.05% to 4.5% copper by weight, as described herein, demonstrated an ohm meter reading of 3 to the power of ten. By contrast, ohm meter tests of other known copper-infused fabrics did not result in measurable ohm meter readings.

In the case of a three layer fabric, yarn position on the three layer fabric may be as follows:
1) All face feeds: copper-containing fiber; all inlay feeds: polymer filaments; and/or all back feeds: polymer filaments.
2) All face feeds: polymer fiber; all inlay feeds: copper-containing fiber; and/or all back feeds: polymer filaments.
3) All face feeds: polymer filaments; all inlay feeds: polymer filaments; and/or all back feeds: copper-containing fiber.

The yarn can be used by itself or with any other known and unknown fibers. Known fibers, such as Celliant, coolmax, cellulose fibers, FR. Fibers, infrared yarn, natural or manmade fiber, either as outer layer or inner layer. The Cu content in the yarn by weight may be between 0.05% to 99%, including between about 0.05% to 4.5% by weight. The Cu content in the yarn by weight may be about 15%, 30%, 45%, 60%, 75% and 95%. Total content of this yarn in the fabric or textile of the present invention may be anywhere between about 8.5% to about 91.2% of the total in the fabric, including 9%, 10%, 15%, 20%, 25%, 30%, 35%, 40%, 45%, 50%, 55%, 60%, 65%, 70%, 75%, 80%, 85%, 90% and any percentage there in between.

The fabric of the present invention can include anywhere from about 3 ppm to about 75 ppm of copper content.

This yarn may be spun or filament and may be used on one layer or in multiple layer fabrics. The fabric can be made using known technologies, such as knitting technology, woven technology or non-woven technology.

The fabric may be made using elastane yarn (i.e. Spendex® or Lycra®).

The copper-containing fabric or textile of the present invention may be manufactured by any method known in the art. In one embodiment, the process for making the copper-containing fabric properties includes:
(a) knitting a fabric on a circular weft knitting machine (or dial selection machine, or any other such type of knitting or weaving machine as may be invented during the term of this patent) using a group of yarns consisting of various fibers or a combination thereof;
(b) introducing a copper thread or yarn to the back of the fabric, referring to the dial in any combination of other fibers; or
(c) introducing a copper thread or yarn to the face of the fabric, referring to the cylinder in any combination of other fibers; or
(d) introducing a copper thread as a filler, referring to the space between the face and the back of the fabric in any combination of other fibers.

The knitting machine may be of various diameters and gauges and a combination thereof. The thread or yarns may be of various colors, counts or deniers and any combination thereof.

The process of manufacture may also include the use of various knitted structures and a combination thereof. Full and half gauge structures are also included and a combination thereof.

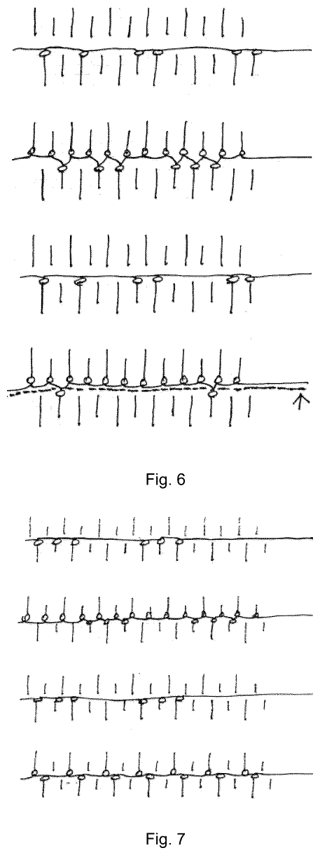

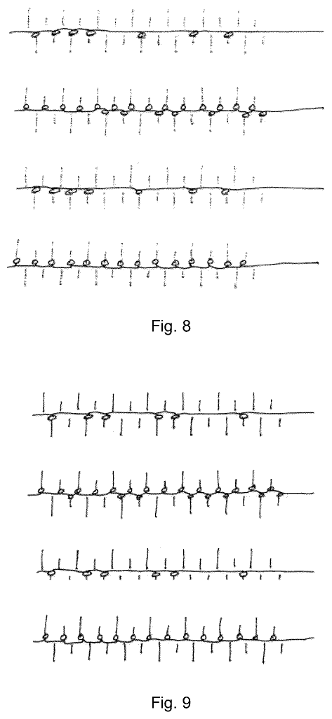

FIGS. 6-9 illustrate non-limiting knitting sequences of a copper-containing fabric according to different embodiments of the present invention.

Tables 1-4 illustrate the knitting sequences for FIGS. 6 to 9 respectively.

TABLE 1

| Feed # | Dial set out | Cylinder set out | Yarn |
|---|---|---|---|
| 1 | Welt all | Select as per design | Spun poly |
| 2 | Knit all | Select as per design | Black tex/Poly |
| 3 | Welt all | Select as per design | Spun Poly |
| 4 | Welt all | Select as per design | Red Tex/Poly + |
| | | | copper inlay* |

*does not knit, the copper is sandwiched between face and back of fabric

TABLE 2

| Feed # | Dial set out | Cylinder set out | Yarn |
|---|---|---|---|
| 1 | Welt all | Select as per design | Tex poly |
| 2 | Knit all | Select as per design | Spun Poly |
| 3 | Welt all | Select as per design | Tex Poly |
| 4 | Knit all | Select as per design | Copper** |

**Copper is knitting on dial and cylinder feed and showing on both face and back of fabric.

TABLE 3

| Feed # | Dial set out | Cylinder set out | Yarn |
|---|---|---|---|
| 1 | Welt all | Select as per design | Cotton |
| 2 | Knit all | Select as per design | Poly/cotton |
| 3 | Welt all | Select as per design | Copper*** |
| 4 | Knit all | Select as per design | Poly/cotton |

***Copper is knitting on cylinder only feed and showing on face only.

TABLE 4

| Feed # | Dial set out | Cylinder set out | Yarn |
|---|---|---|---|
| 1 | Welt all | Select as per design | Spun poly |
| 2 | Knit all | Select as per design | Tex/Poly |
| 3 | Welt all | Select as per design | Spun Poly |
| 4 | Knit all | Select as per design | Copper**** |

****Copper is knitting on dial only feed and showing on back only.

Alternatively, the fabric containing copper of the present invention may include a woven or non-woven textile that has been impregnated in or coated with copper. Jeffrey Gabbay et al. (J. of Industrial Textiles, vol. 35, issue 4, 2006, p. 323, the contents of which are incorporated herein by reference) provide an example on impregnating textiles with copper oxide. “Briefly, cotton fibers are plated as follows: cationic copper (a mix of Cu²⁺ and Cu¹⁺) is bound to cellulose-based fibers. Cotton, rayon/viscose, and fibers such as lyocel by Tencel are treated with an electroless plating process, which includes the following steps: (a) fibers having a diameter of about 11-13 mm are soaked for 5 s in 1% SnCl2, pH 3.5 at room temperature; (b) the fibers are then soaked for 5 s in PdCl2, pH 4 at room temperature, producing activated fibers; and (c) the activated cellulose based fibers are then exposed to formaldehyde, CuSO₄, and polyethylene glycol at pH 9. After about 5 min the fibers are plated with cationic copper (Cu(II) and Cu(I)). Finally, the fibers are dried and run through a textile carding machine which separates and aligns them.

“Impregnation of copper into the various synthetic fibers mentioned above is achieved by adding a cupric oxide powder to the polymers during the master batch preparation stage. The master batch can be made in industrially accepted concentrations and added to the polymeric slurry the same way any other master batch would be added, such as for pigmentation, etc. The copper oxide doped master batch is designed in such a way as to allow fiber extrusion in the normal production systems. The fibers can be cut into short staple or produced in filament form and texturized, if so desired. As in the case of the plated fibers mentioned above, the product yielded is a fiber that can be introduced at the blending stage of yarn production or directly into a woven or knit product so that no manufacturing processes are changed.” (See Jefferey Gabbay et al (supra) at p. 325).

The articles of manufacture of the present invention, including foam, fabric or textile may have different applications, including (i) top of the bed product, (ii) mattress and mattress covers, (iii) removable mattress toppers, (iv) pillow cases, (v) pillow shell, (vi) pillow fill, (vii) blanket and blanket fill, (viii) fitted and loose sheets for bedding, (xi) mattress pads, (xii) chair liners, (xiii) sleeping bags, (xiv) clothing, including pajamas, socks and so forth, (xv) towels, (xvi) in hospital settings in mattress/toppers/removable covers/all related to mattress or any resting or sleeping surface, (xvii) in mattresses and/or (xviii) in tents. The articles of manufacture of the present invention may also include items such as powders and soaps containing copper.

The articles of the present invention may be labelled for its properties. For example, the articles may include a label stating that it can reduce, prevent and/or treat one or more of the health conditions listed in this document.

One advantage of the copper-containing articles of the present invention is that they may be administered to a subject to treat/prevent or ameliorate a condition in a natural, non-invasive, pain-free, non-chemical, earthing way.

### Therapeutic Uses

The copper-containing articles of the present invention can be used in a number of therapeutic and preventive methods.

A method for controlling or managing body weight is provided. The method includes exposing a subject to an article containing copper.

A method for improving insulin sensitivity is provided. The method includes exposing a subject in need of improving insulin sensitivity to an article containing copper.

A method for improving cardiovascular health is provided. The method includes exposing a subject to an article containing copper.

A method for increasing the amount or promoting the growth of beneficial bacterial species in the GI tract of a subject, the method including exposing the subject to an article containing copper. The beneficial bacteria include Lactobacillus sp. (LB); Clostridium coccoides sp (CC); and Segmented filamentous bacteria (SFB). The method can promote the growth of bifido- and lacto-bacteria in the gastro-intestinal tract at the expense of pathogens such as Clostridium perfringens. This method may be beneficial for subjects having, for example, leaky gut to reduce the amount of non-beneficial, toxic bacteria from entering the blood stream. As such, the method of increasing the amount or promoting the growth of beneficial bacteria of the present invention may be used to prevent or treat inflammatory bowel diseases, type 1 and type 2 diabetes, asthma, multiple sclerosis, Hashimoto's disease, and other autoimmune disorders.

The copper containing articles used can be an article of manufacture containing copper. The article or article of manufacture may contain an effective amount of copper.

In order to aid in the understanding and preparation of the within invention, the following illustrative, non-limiting, examples are provided.

### 6. EXAMPLES

### 6.1 Materials

Animals: male c57bl/6j mice.

Diet: high fat high cholesterol diet from Open source diets -D12079B.

Protocol: The animals were fed the high fat high cholesterol (HFHC) diet for 2 months and were housed in fabric lined cages; a) fabric with copper coating or b) the same fabric without copper coating.

Objectives: This study was designed to reveal whether copper-containing fabrics can help mice with diet-induced obesity improve their metabolic and cardiovascular health condition.

Male mice were fed the HFHC for a period of 8 weeks to induce obesity (6 mice per group) with/without copper-infused fabric beddings to generate direct data to provide evidence on the role of the copper-containing fabric products on the improvement of health conditions.

### 6.2 Results

### 6.2.1 Metabolic Status

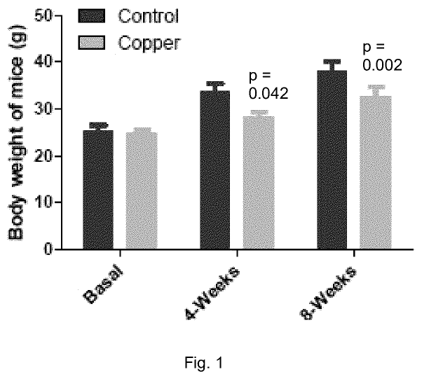

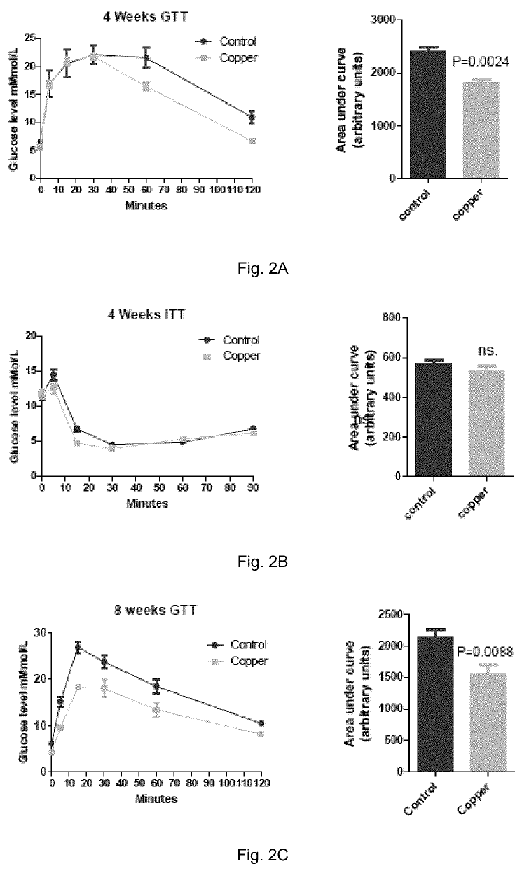

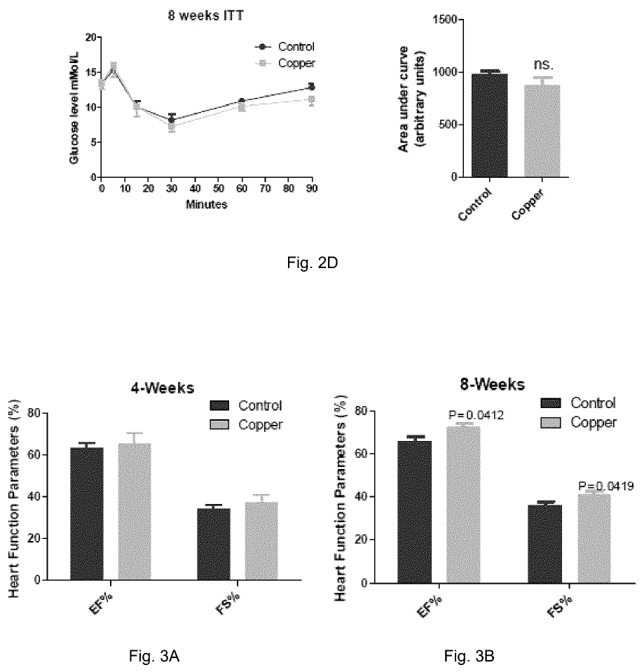

At the beginning and then monthly during the protocol the body weight, circulating insulin and glucose levels were examined. Glucose tolerance tests and insulin tolerance tests to determine changes in peripheral insulin sensitivity and metabolism were performed. The results demonstrate that mice treated with copper-infused liner had less body weight increase (see FIG. 1) and better insulin sensitivity (see FIGS. 2A-2D) than control mice.

### 6.2.2 Cardiovascular Status

Echocardiography was performed on the mice monthly until 8 weeks period. Images were obtained using the Vevo2100 (Visual Sonics, Toronto, ON, Canada) equipped with an MS550D transducer. The mice were lightly anaesthetized using 1.5% isofluorane mixed with 100% O2 and the M-mode image was acquired when the body temperature (36.9-37.2 Celsius) and respiration rate (100) were stable and within a proper range during the time of imaging. M-mode images of the parasternal short-axis view at papillary level were used to calculate the cardiac systolic functions of ejection fraction and fractional shortening. Analysis was performed using VevoStrain software with recordings acquired from the M-mode view. All parameters were averaged over at least 3 cardiac cycles for analysis.

The results showed that heart functions are improved significantly in mice treated with copper-infused liner under HFHC challenge compared to those housed in fabric without copper coating (see FIGS. 3A and 3B).

### 6.2.3 Gut Microbiota Status

Mammals harbor a complex gut-associated microbiota, comprising bacteria that provide immunological, metabolic and neurological benefits to the host, and contribute to their wellbeing. However, dysregulation of the microbiota composition, known as dysbiosis, has a key role in the pathogenesis of many inflammatory diseases, including inflammatory bowel diseases, type 1 and type 2 diabetes, asthma, multiple sclerosis, and so on.

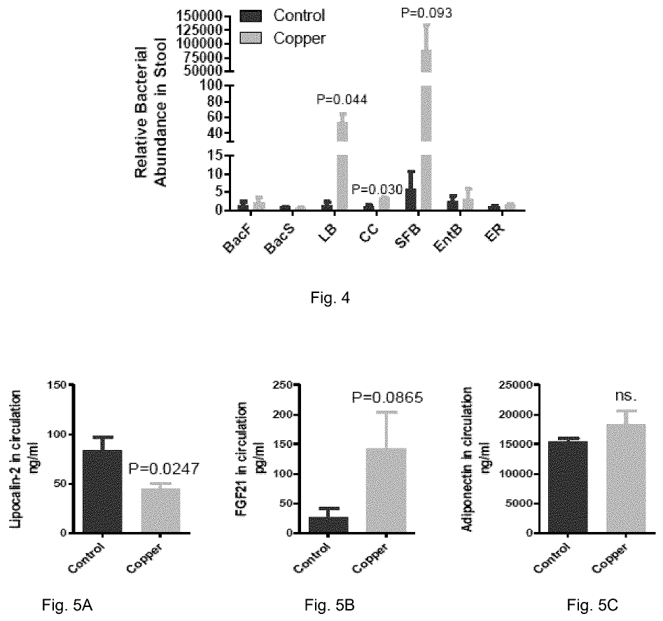

In the present study, seven (7) phyla of the gut microbiota were analyzed and found that mice with copper-containing liner treatment boosts the abundance of bacterial family such as Lactobacillus (LB), Clostridium (CC) and Segmented filamentous bacteria (SFB) in mice under HFHC challenge (see FIG. 4). On the other hand, no boost in abundance was seen for B. fragilis, Bacteroides sp., Enterobacteriaceae and Eubacterium rectale. This data suggests that copper fabric primarily elevates bacteria which are beneficial for health.

Interestingly, copper has been shown in the past to have anti-microbial properties. Therefore the use of articles containing copper to promote the growth of beneficial bacteria, or food-grade bacteria, is unexpected.

The simple way to consider these data are that some gut bacteria are known to improve health (for example, these are the focus of probiotics) and others have a bad or deleterious influence on body health.

### 6.2.4. Cardiometabolic Status

Various blood-borne diagnostic markers of inflammation and adipose tissue dysfunction characterize the HFHC model. Accordingly, blood samples were taken at the end of experimental protocol and measured the following biomarkers:
a) the well-known pro-inflammatory marker lipocalin-2 is significantly increased by HFHC but mice housed in copper fabric did not have this increase.
b) FGF-21 is significantly increased and adiponectin is slightly increased in blood of mice treated with copper-infused liner (see FIG. 5) and this could be relevant because FGF-21 and adiponectin are both known to be good for health. Many pharmaceutical companies have tried to develop drugs targeting FGF-21 and adiponectin.

### 6.3 Conclusion

In response to HFHC, mice with copper-infused fabric liner had
Less body weight gain
Improved insulin sensitivity and thus better glucose handling (less diabetes)
Improved heart function
Dynamic changes in the gut microbiome
Less circulating lipocalin-2
Enhanced levels of circulating FGF-21 and adiponectin

Collectively these data indicate significantly beneficial body health.

In other tests, studies using 4 layers of fabric, where the bottom layer contained copper, demonstrated effects on bacteria on the top layer fabric, which did not contain copper.

In other tests, studies using rats that slept on bedding containing copper, the rats exhibited increases in the amount of adiponectin or FGF-21 and reduction in the amount of lipocalin-2 in blood. These results can be explained by the release of copper ions from the bedding containing copper, and the copper ions travelling through the cutaneous layer to exert the effects.

Those skilled in the art will recognize, or be able to ascertain using no more than routine experimentation, many equivalents to the specific embodiments of the invention described herein. Such equivalents are intended to be encompassed by the following claims.

What is claimed is:

1. A method of increasing the amount of adiponectin or FGF-21, or of reducing the amount of lipocalin-2, in blood, the method comprising contacting a subject with an article containing copper wherein the article containing copper comprises between 0.05% to 4.5% copper by weight and is capable of releasing copper ions, and wherein the release of copper ions increases the amount of adiponectin or FGF-21, or reduces the amount of lipocalin-2, in blood.

2. The method of claim 1, wherein the subject is a human.

3. The method of claim 1, wherein the article containing copper, is a liquid treated article, wherein the liquid contains copper.

4. The method of claim 1, wherein the article containing copper, is a copper infused article.

5. The method of claim 1, wherein the copper-containing article is a copper-containing article of manufacture.

6. The method of claim 1, wherein the copper-containing article is a fabric.

7. The method of claim 6, wherein the fabric comprises a single layer.

8. The method of claim 6, wherein the fabric comprises 2 or more layers, and wherein the copper is contained in the layer that is not in contact with the subject.

9. The method of claim 6, wherein the fabric comprises 3 or more layers, the three or more layers comprising a face layer, an inlay and a back layer, wherein the inlay and the back layer is not in contact with the subject, and wherein the copper is contained in the inlay or the back layer that is not in contact with the subject.

10. The method of claim 6, wherein the fabric is selected from a sheet, top of the bed product, mattress and mattress covers, removable mattress toppers, ticking fabric for covering a mattress pillow cases, pillow shell, pillow fill, blanket and blanket fill, fitted and loose sheets for bedding, mattress pads, chair liners, clothing, towels and sleeping bag.

11. A method for promoting the growth of beneficial bacteria in the gastro-intestinal tract of a subject comprising contacting the subject with an article containing copper, wherein the article containing copper comprises between 0.05% to 4.5% copper by weight and is capable of releasing copper ions, and wherein the release of copper ions promotes the growth of beneficial bacteria in the gastro-intestinal tract of the subject.

12. The method of claim 11, wherein the beneficial bacteria are probiotic bacteria.

13. The method of claim 11, wherein the beneficial bacteria include Lactobacillus sp. (LB), Clostridium coccoides sp (CC), and Segmented filamentous bacteria (SFB).

14. A method of managing the body weight of a subject, comprising contacting the subject with an article containing copper, wherein the article containing copper comprises between 0.05% to 4.5% copper by weight and is capable of releasing copper ions.

15. The method of claim 14, wherein the method further includes the steps of weighing and recording a body weight of the subject before and after exposure to the article containing copper and comparing the body weight before and after exposure.

16. A method for improving or increasing insulin sensitivity, or for promoting, treating and/or prophylaxis of cardiovascular health, in a subject in need thereof, the method comprising contacting the subject with an article containing copper, wherein the article containing copper comprises between 0.05% to 4.5% copper by weight and is capable of releasing copper ions.

17. The method of claim 16, wherein the subject suffers from type 2 diabetes.

18. The method of claim 1, wherein the article containing copper, is a copper infused article.

19. The method of claim 1, wherein the copper-containing article is a fabric.

20. The method of claim 1, wherein the fabric comprises 2 or more layers, and wherein the copper is contained in the layer that is not in contact with the subject.
